(19) United States
(12) Patent Application Publication
(10) Pub. No.: US 2022/0323116 A1
(43) Pub. Date: Oct. 13, 2022

(54) CERVICAL FRICTION PLATE

(71) Applicant: ORTHOPAEDIC EDUCATION AND RESEARCH INSTITUTE D/B/A HOAG ORTHOPEDICS, Orange, CA (US)

(72) Inventors: Vance GARDNER, Irvine, CA (US); Gerald ALEXANDER, Irvine, CA (US); Matthew DUFFIN, Charlotte, NC (US); Robert GORAB, Newport Beach, CA (US); Ryan HELBER, Dana Point, CA (US); Oluwadamilola OLUYEDE, Bakersfield, CA (US); Edward QUILLIGAN, Huntington Beach, CA (US); Carlos PRIETTO, Laguna Beach, CA (US); Jeremy SMITH, Laguna Beach, CA (US); Jon WHITE, Laguna Beach, CA (US)

(21) Appl. No.: 17/731,725

(22) Filed: Apr. 28, 2022

Related U.S. Application Data
(63) Continuation-in-part of application No. 17/714,980, filed on Apr. 6, 2022.
(60) Provisional application No. 63/181,142, filed on Apr. 28, 2021.
(60) Provisional application No. 63/171,910, filed on Apr. 7, 2021.

(51) Int. Cl. A61B 17/70; A61B 17/80

(52) U.S. Cl. CPC A61B 17/7059; A61B 17/809

(57) ABSTRACT

Disclosed are devices, systems and related surgical methods for improving the fixation and/or durability of bone implants and/or components thereof for cervical fracture fixation and/or other orthopedic procedures.

### CROSS-REFERENCE TO RELATED APPLICATIONS

This application is a continuation-in-part of U.S. Utility patent application Ser. No. 17/714,980 entitled “ORTHOPAEDIC FRICTION PLATE,” filed Apr. 6, 2022, which claims the benefit of U.S. Provisional Application No. 63/171,910 entitled “ORTHOPAEDIC FRICTION PLATE,” filed Apr. 7, 2021. This application further claims the benefit of U.S. Provisional Application No. 63/181,142 entitled “CERVICAL FRICTION PLATE,” filed Apr. 28, 2021. Each of these above-referenced disclosures are hereby incorporated by reference herein in their entireties.

### FIELD OF THE INVENTION

The present subject matter relates generally to surgical devices and more specifically to devices, system and methods for spinal fusion, fracture fixation and/or other orthopedic procedures of the cervical spine.

### BACKGROUND OF THE INVENTION

Many bony fractures require stabilization that cannot be provided by external splints or casts—internal fixation is therefore required. Bone plates are among the most common artificial orthopedic implants and are commonly used to stabilize and internally fixate bony fractures, as well as to provide support to and/or bridge between bones, such as bones of the cervical spine during spinal stabilization, fixation and/or arthroplasty procedures.

With regards to the spine of humans, internal fixation using mechanical hardware is often the gold standard for repair of injured and/or fractured spinal bones and related structures. There are 7 cervical spinal vertebrae, and in the cervical spine vertebrae 4 though 7 are connected to each other by an anterior (front side) cartilaginous intervertebral disc and a posterior (back side) connection called the facet joint (i.e., there are 2 facet joints between each vertebrae on the posterior spine—left and right). A functional spinal “unit” can comprise two vertebrae that are connected at one disc and 2 facet joints, which unit is commonly referred to as a “motion segment”. A disc space between adjacent vertebral bodies is typically called the “interbody” region, with a fusion occurring through this space called an “interbody fusion”. A fusion can also span an entire vertebral body(s) if the body is needed to be surgically removed (called a corpectomy). In a corpectomy, a plate may be fixed to one or more vertebrae above and below the reconstructed vertebral body.

A spinal fusion is a surgical procedure which seeks to address and provide skeletal stability following a vertebral fracture or instability due to tumor or infection or reconstruction following removal of a herniated disc or bone material that is impinging a spinal nerve or the spinal cord. Bone graft, whether from the patient's own bone (autologous), or from donated cadaver bone (allograft bone) is often needed for the fusion. In such procedures, the bone can be incorporated within a device that provides structure to the grafting material. The device is sometimes called an “interbody cage” if an interbody fusion in needed, or if the device replaces the entire vertebral body(s) that is being reconstructed, it may be called a “vertebral or corpectomy cage”.

In modern spine surgery, the creation of stable anterior cervical spine fixation can be a desirable approach, thereby creating an anterior fusion (or arthrodesis) of one cervical vertebrae to another. Vertebral stabilization can optimally be achieved through instrumentation of the spine, such as the application of one or more fusion plates and associated fixation hardware. In the cervical spine, a fixation plate can be is placed on an anterior (i.e., frontal) aspect of the vertebral bodies, and then secured to the vertebral bodies using 2 or more screws for fixation. The plate can be manufactured with a coronal curvature to provide optimal plate bone interaction. The plate can similarly be manufactured with a sagittal curve, or it can be contoured into a desired configuration and/or lordosis at surgery. A plate may span an intervertebral disc segment that has been prepared for fusion (or arthrodesis) and attaches to the next distal vertebrae. Preparation for fusion can be performed by removing the intervertebral disc and replacing it with either allograft or autograft bone graft, a fusion implant (such as an intervertebral “cage”), or both. This type of vertebral fixation for intervertebral fusion can be a single level or multiple levels, with each vertebral level requiring 2 screws fixing the plate to each per level.

In other surgeries, instead of just an intervertebral fusion requiring fixation of the vertebrae, an entire vertebra (or series of vertebrae) may be removed (vertebrectomy). Stabilization of such a defect can require preparation for fusion to the vertebrae proximal or distal (above and below) the vertebrectomy level(s), either with bone graft alone or an intervertebral fusion device. The plate fixation then may span the entire length of the removed vertebrae and desirably fixates to the intact vertebra above and below the vertebrectomy level(s)

Various disease states requiring fusion and plate fixation (either after intervertebral disc fusion or fusion after vertebrectomy) can include degenerative disc disease, spinal fracture, tumor, deformity, infection, inflammatory (spondyloarthropathies) or any process that requires removal of disc or bone, creating an unstable spinal segment(s) that requires stable, anterior plate fixation for the reconstruction.

A conventional bone plate is essentially a rigid metal plate drilled with guide holes through which bone screws can be passed. Bone screws are usually inserted through the mounting holes and threaded into the bone above and below the fracture to fix the bone plate, thereby stabilizing and fixating the fracture. In some cases, the bone plate may be removed after healing—although in many cases the plate and associated fixation devices are left permanently implanted.

In traditional plates, a bone plate is pressed onto the bone surface by one or more screws, with the tightening torque of the bone screw typically determining the level of securement and/or “friction” between the plate and bone. While this may lead to a well secured plate at the time of surgical implantation, healing, bone remodeling and/or cyclic loading of the plate and/or bone screws can lead to undesirable loosening of the bone plate and/or displacement of bone fragments. Moreover, the large, required contact surfaces of some bone plates can disturb and/or interrupt blood circulation in treated bone regions. Moreover, in some scenarios a fusion across a given spinal level may not fully occur heal (called a pseudarthrosis), and the mechanical fusion construct can fail over time. In that situation, the plate can break, a screw can break, or a screw can back out, or all of these modes of failure can occur. Failure of the fixation and fusion usually requires more surgery to alleviate the problem.

Accordingly, there is need for further improvement in bone plate systems and related surgical implants, and the present subject matter is such improvement.

### BRIEF SUMMARY OF THE INVENTION

The following presents a simplified summary of the subject matter in order to provide a basic understanding of some aspects of the subject matter. This summary is not an extensive overview of the subject matter. It is intended to neither identify key or critical elements of the subject matter nor delineate the scope of the subject matter. Its sole purpose is to present some concepts of the subject matter in a simplified form as a prelude to the more detailed description that is presented later.

In accordance with various aspects of the present subject matter, systems, devices and/or methods are disclosed which may be useful in bone and/or joint repair procedures or other orthopedic procedures. Various embodiments can include enhancing orthopedic surgical plates to increase a force of resistive friction at the bone plate interface in order to create a more stable construct, desirably for anterior cervical spine plates. Such implants can be designed to fixate vertebral bodies adjacent to a disc space that is being “fused” with an interbody bone graft or cage and bone graft construct. Fixation of the vertebral bodies may be achieved through two screws (left and right) placed through the plate (from Anterior to Posterior) into each vertebral body that is part of the fixation and fusion construct. In various embodiments, a “textured” surface can be created and placed in contact with bony surfaces in order to enhance the coefficient of friction between the plate and the bone in order to limit motion and increase stability of fixation constructs.

In various applications, the disclosed implant components can comprise various medical materials, including the use of one or various combinations of titanium, chrome cobalt, stainless steel, silicone, poly (ether ether ketone) (PEEK), ultra-high molecular-weight polyethylene (UHMWPE), polylactic acid, apatites and/or various 3D printable materials. Where a plurality of different materials may be used in a single implant, the employment of mixed materials in the implant construction may enhance the strength and/or durability of a desired implant design, as well as allow for improved surgical outcomes and/or greatly reduced complication rates.

If desired, implant components could be constructed from a variety of modular components, including modular components comprising different materials. If desired, such modular components could be provided in a kit form for selection and/or assembly in a surgical theatre and/or in situ during a surgical procedure. If desired, various components may be removable and replaceable.

Disclosed herein are a variety of implant components that can be utilized during an orthopedic surgical procedure to repair and/or stabilize an injured or diseased bone region of a patient. Various surgical methods for assembling such implant components and/or for implanting or placement of the various devices and/or components described herein are also described, including the insertion and placement of implants between and/or along fractured regions of a long bone, and within bones and/or between bones or joint surfaces.

In accordance with another aspect of the present subject matter, various methods for manufacturing devices and/or components thereof, as set for within any of the details described with the present application, are provided and/or contemplated herein.

While embodiments and applications of the present subject matter have been shown and described, it would be apparent that other embodiments, applications and aspects are possible and are thus contemplated and are within the scope of this application.

The following description and the annexed drawings set forth in detail certain illustrative aspects of the subject matter. These aspects are indicative, however, of but a few of the various ways in which the principles of the subject matter may be employed and the present subject matter is intended to include all such aspects and their equivalents. Other objects, advantages and novel features of the subject matter will become apparent from the following detailed description of the subject matter when considered in conjunction with the drawings

Various surgical methods for preparing anatomical surfaces and/or for implanting or placement of the various devices and/or components described herein may also be described, including the insertion and/or placement of implants between and/or along damaged and/or diseased regions of a patient's long bone.

### BRIEF DESCRIPTION OF THE SEVERAL VIEWS OF THE DRAWINGS

The foregoing and other features and advantages of the present subject matter will become apparent to those skilled in the art to which the present subject matter relates upon reading the following description with reference to the accompanying drawings.

FIG. 1 depicts one exemplary embodiment of a bone plate for splinting a fracture on a bone;

FIG. 2 depicts an enlarged bone contacting surface of another exemplary embodiment of a bone plate;

FIG. 3 depicts an enlarged bone contacting surface of another exemplary embodiment of a bone plate;

FIG. 4 depicts an orthopedic plate secured to a long bone by a plurality of bone screws;

FIGS. 5A and 5B depict an exemplary securement of a bone plate to a bone surface;

FIG. 5C depicts an alternative securement of a bone plate where the bone surface presents a rough or irregular surface;

FIG. 6 depicts an exemplary series of plate and screw movements relative to a bone plate that can result in screw toggle and eventual screw “pullout” from the plate due to motion at the plate/bone interface;

FIGS. 7A and 7B depict various forces acting on a screw/plate interface;

FIG. 8 graphically depicts the linear relationship between the Force of static friction and both the Coefficient of static friction and the Normal force;

FIGS. 9A and 9B depict an exemplary static bone plate;

FIG. 10A depicts a smooth bone plate manufactured using 3D printing;

FIG. 10B depicts a textured bone plate manufactured using 3D printing

FIG. 11 graphically depicts a motion resistance of both the textured plate and smooth plate on a similar bone surface analog;

FIG. 12A depicts another exemplary embodiment of textured plates, with two plates secured to a test fixture;

FIG. 12B graphically depicts the results of the dynamic testing model of FIG. 12A, with the textured plate results at the top of the graph;

FIG. 13 depicts one exemplary embodiment of a textured bone plate analog having an exemplary LC-DCP screw hole;

FIG. 14A and 14B depict characteristics of various different surface texture embodiments which were developed, constructed and tested against equivalent sized smooth plates to determine values for a coefficient of friction;

FIG. 15A depicts another exemplary experiment comparing plates with the addition of bone facing textured surfaces against smooth surfaced plates;

FIG. 15B graphically depicts various results from the experiment of FIG. 15A;

FIGS. 15C and 15D depict various increases of the coefficient of friction and increased force of friction for textured plates;

FIGS. 15E and 15F graphically depict various results from the experiment of FIG. 15A;

FIG. 16 depicts another exemplary embodiment of a surgical plate for use with various surgical procedures;

FIGS. 17A through 17C depict various views of another alternative embodiment of a surgical plate for use with various surgical procedures;

FIGS. 18A and 18B depict views of another alternative embodiment of a surgical plate for use with various surgical procedures;

FIGS. 19A through 19C depict views of another alternative embodiment of a surgical plate for use with various surgical procedures;

FIGS. 20A through 20C depict views of another alternative embodiment of a surgical plate for use with various surgical procedures;

FIG. 21 depicts exemplary embodiments of pyramidal-shaped protrusions and elongated wave or tread-shaped protrusions; and

FIG. 22 depicts results from an exemplary testing comparison between a smooth plastic plate and a textured plastic plate.

The following description and the annexed drawings set forth in detail certain illustrative aspects of the subject matter. These aspects are indicative, however, of but a few of the various ways in which the principles of the subject matter may be employed and the present subject matter is intended to include all such aspects and their equivalents. Other objects, advantages and novel features of the subject matter will become apparent from the following detailed description of the subject matter when considered in conjunction with the drawings.

### DETAILED DESCRIPTION OF THE INVENTION

The disclosure and the various features and advantageous details thereof are explained more fully with reference to the non-limiting embodiments and examples that are described and/or illustrated in the accompanying drawings and detailed in the following description. It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments of the disclosure. The examples used herein are intended merely to facilitate an understanding of ways in which the disclosure may be practiced and to further enable those of skill in the art to practice the embodiments of the disclosure. Accordingly, the examples and embodiments herein should not be construed as limiting the scope of the disclosure. Moreover, it is noted that like reference numerals represent similar parts throughout the several views of the drawings.

The terms “including,” “comprising” and variations thereof, as used in this disclosure, mean “including, but not limited to,” unless expressly specified otherwise. The terms “a,” “an,” and “the,” as used in this disclosure, mean “one or more,” unless expressly specified otherwise.

Devices and/or device components that are disclosed in communication with each other need not necessarily be in continuous communication with each other, unless expressly specified otherwise. In addition, devices that are in direct contact with each other may contact each other directly or indirectly through one or more intermediary articles or devices.

Although process steps, method steps, or the like, may be described in a sequential order, such processes and methods may be configured in alternate orders. In other words, any sequence or order of steps that may be described does not necessarily indicate a requirement that the steps be performed in that order. The steps of the processes or methods described herein may be performed in any order practical. Further, some steps may be performed simultaneously.

When a single device or article is described herein, it will be readily apparent that more than one device or article may be used in place of a single device or article. Similarly, where more than one device or article is described herein, it will be readily apparent that a single device or article may be used in place of the more than one device or article. The functionality or the features of a device or article may be alternatively embodied by one or more other devices or articles which are not explicitly described as having such functionality or features.

Certain exemplary embodiments will now be described to provide an overall understanding of the principles of the structure, function, manufacture, and use of the devices and methods disclosed herein. One or more examples of these embodiments are illustrated in the accompanying drawings. Those of ordinary skill in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

The present invention provides various devices, systems and methods for treating various anatomical structures of bones and/or other areas of human and/or animal bodies. While the disclosed embodiments may be particularly well suited for use during surgical procedures for the repair, fixation and/or support of the cervical spine, it should be understood that various other anatomical locations of the body may benefit from various features of the present invention.

In various disclosed embodiments, a primary objective of the disclosed invention can be to increase the resistance of a bone plate to various types of sliding motion relative to the bone surface, for both conventional, non-locked plates as well as locked plates, which will desirably create more stable fixation constructs and prevent unwanted motion in a targeted anatomical region. In various embodiments, this will desirably preserve an anatomic reduction and/or preserving the blood supply of the treated region, while providing for an early and safe mobilization of the affected anatomy and/or promote long term fusion of the treated joint.

In fracture fixation, creating a stable construct is generally desirable to allow the broken bones, bone fragments and/or adjacent bones to reattach and/or heal. In some scenarios, if there is excessive motion between the elements of the fixation construct and/or the treated anatomy, the bones will not heal or fuse and/or the construct fails. In many examples, fracture fixation with construct failure can occur where the screws and/or plates have lost “purchase,” and in some cases, the hardware itself has broken. Such failures can include screw toggle; screw backout; fatigue, strain and/or work hardening of various components, plate bending and/or fracture, various combinations thereof and/or a variety of other failure modes.

In some instances, commercially available fixation plates and/or related components might benefit from modification in light of the inventions disclosed herein, and the modification of such plates to incorporate the concepts of the present intention are contemplates herein. Such modifications may include a variety of steps, which can include the addition and/or removal of material to create a roughened or textured bone facing surface proximate to one or more fixation devices or securement locations on the plate. One exemplary embodiment of a commercially available implant system that could greatly benefit from the various teachings provided herein is the Aviator® Anterior Cervical Plating System, commercially available from Stryker Spine of 2 Pearl Court, Allendale, N.J., USA). A brochure describing the various system components and related surgical technique and related instruments is publicly available on the internet at http://az621074.vo.msecnd.net/syk-mobile-content-cdn/global-content-system/SYKGCSDOC-2-37679/gfiboWGyHVpuzaUaxMRBg1GaNALRAA/CVAVI_ST_1pdf, the disclosure of which is incorporate by reference herein in its entirety.

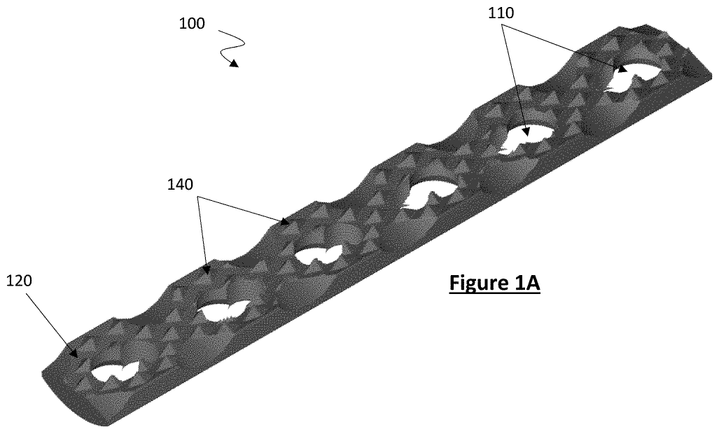

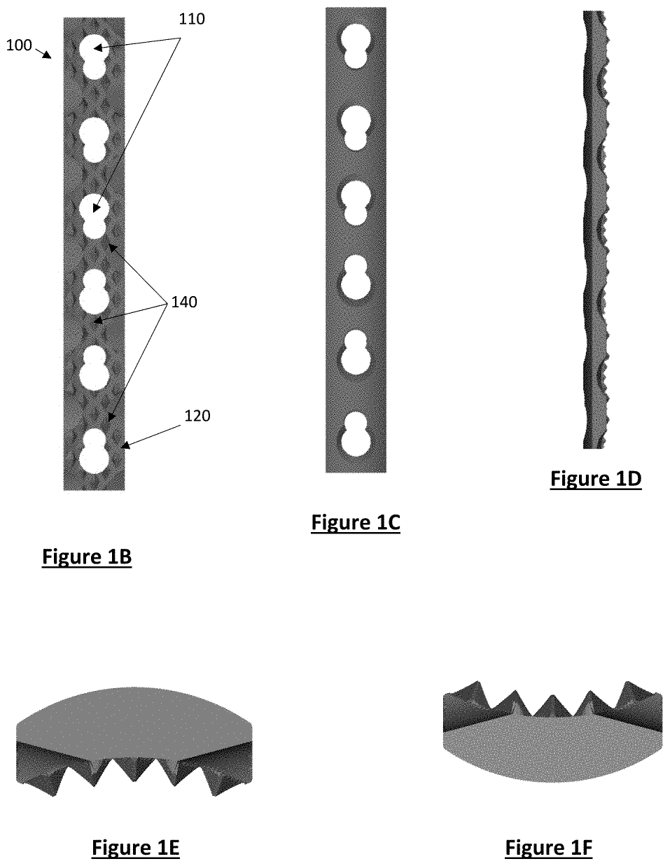

FIG. 1 depicts one exemplary embodiment of a bone plate 100 for splinting a fracture on a bone. The bone plate 100 includes a plurality of bone screw openings 110, with a bone contacting surface 120 of the plate including a roughened or textured bone contacting surface 130, which in this case comprises a plurality of protrusions 140 extending outward from the bone contacting surface 130. The plurality of protrusions 140 can include structures, surfaces and/or portions thereof that interact with the bone to desirably increase securement between the plate and the bone. Such securement can include the use of one or more bone screws (not shown) which desirably draw one or more surfaces of the plate into intimate contact the one or more surfaces of the bone.

FIG. 2 depicts an enlarged bone contacting surface 210 of another exemplary embodiment of a bone plate 200, wherein the protrusions 220 comprise a series of pyramid shaped structures extending outward from the surface 210. While the protrusion in this embodiment are of similar shaped and/or sized pyramids, in alternative embodiments the protrusions may be of similar shapes but different sizes, or alternatively may be of similar sizes but different shapes, or in even other alternative embodiments may be of different sizes and different shapes on the same bonne contacting surfaces. In still other embodiments the protrusions may be positioned in aligned, repeating rows of similar orientation, while in other embodiments the alignment, positioning, spacing and/or orientation of adjacent protrusions (or any combinations thereof) may be different.

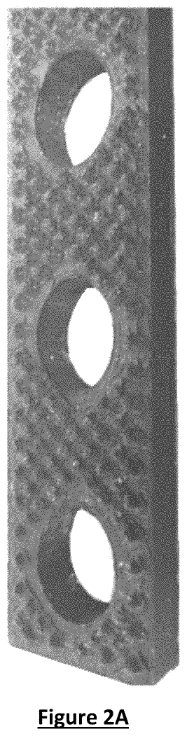

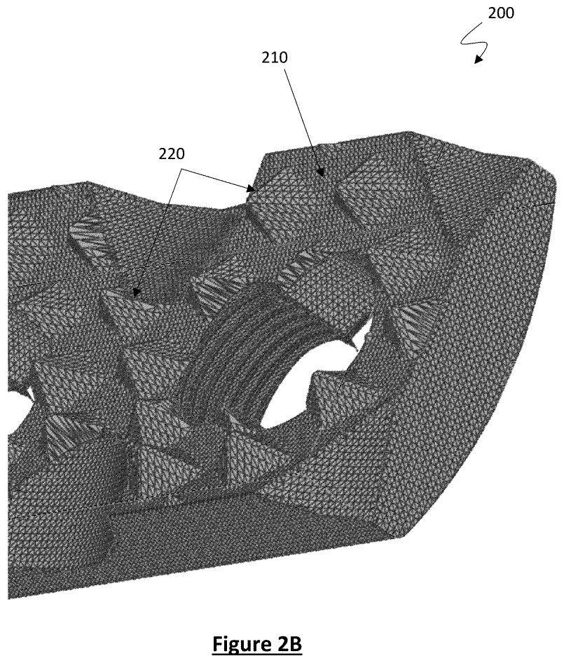

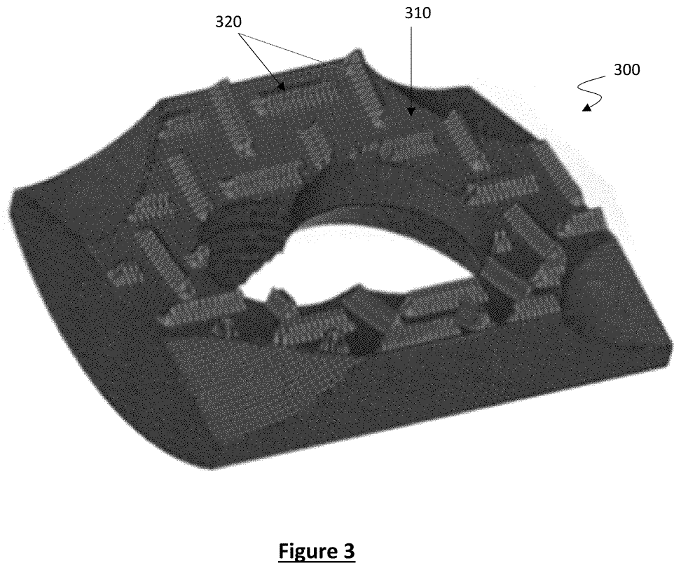

FIG. 3 depicts an enlarged bone contacting surface 310 of still another exemplary embodiment of a bone plate 300, wherein the protrusions 320 comprise a series of elongated tent or peaked structures extending outward from the surface 310. In a similar manner to the previous embodiment, while the protrusions in this embodiment are of similar shaped and/or sized structures, in alternative embodiments the protrusions may be of similar shapes but different sizes, or alternatively may be of similar sizes but different shapes, or in even other alternative embodiments may be of different sizes and different shapes on the same bonne contacting surfaces. While the protrusions of this embodiment are positioned in varying orientations, on other embodiments the protrusions may be positioned in aligned, repeating rows of similar orientations, or the alignment, positioning, spacing and/or orientation of adjacent protrusions (or any combinations thereof) may differ.

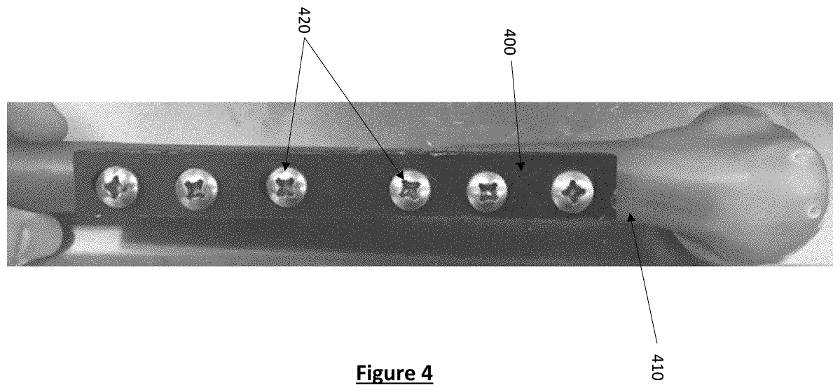

FIG. 4 depicts an orthopedic plate 400 secured to a long bone 410 by a plurality of bone screws 420. Because the plate 400 desirably incorporates a plurality of bone screw openings, a physician may decide to utilize one or more of the openings to accommodate one or more bone screws to secure the plate to the desired anatomical structures. It should be understood that the physician need not necessarily utilize all of the bone screw openings for securement screws and/or bone fixation in a given procedure.

FIGS. 5A and 5B depict an exemplary securement of a bone plate 500 to a bone surface 590. Desirably, the bone plate 500 will include a series of projections 510 or other roughened surface features which face towards the bone surface. Once the bone plate 500 approaches and contacts the bone surface 590 (such as when the surgeon advances and/or tightens bone screws extending through the bone plate into the bone), some portions of the projections will desired contact, penetrate and/or interdigitate with the underlying tissues and/or the bone surface (see FIG. 5B). This will desirably secure the bone plate 500 to the bone surface 590, and desirably reduce and/or eliminate transverse, lateral and/or sliding movement of the plate relative to the bone, as well as various other modalities of movement (i.e., rotational torsion and/or wobbling or toggling). If desired, the surgeon may decide to further advance the bone screws at any point during the implantation surgery, which can increase the Normal force and draw the projections further into the bone surface. In various embodiments, while some portion of the projections may contract and/or penetrate the bone surface, some other base portions 520 of the projections and/or various planar surface portions 530 of the bone plate may not directly contact the bone surface. FIG. 5C depicts an alternative securement of a bone plate, wherein the bone surface may present a rough or irregular surface to the bone plate, with some portion of the projections interdigitating with various of the surface irregularities. In such a case, some of the projections may interlock or interdigitate with various surface features, while other projections may penetrate the surface of the bone, which may provide even more resistance to movement between the plate and the bone.

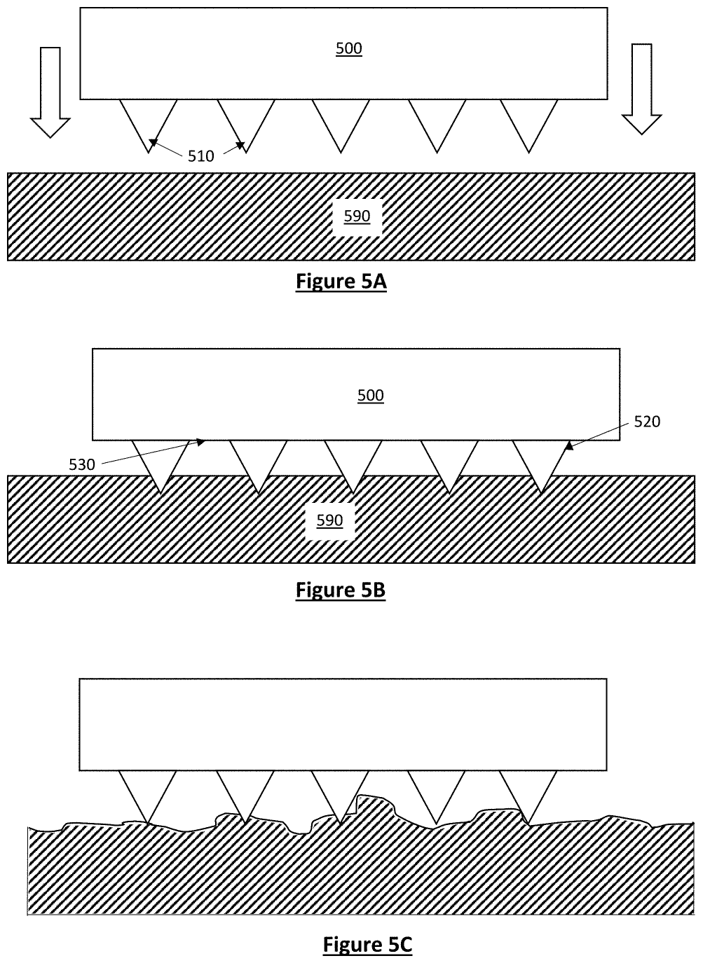

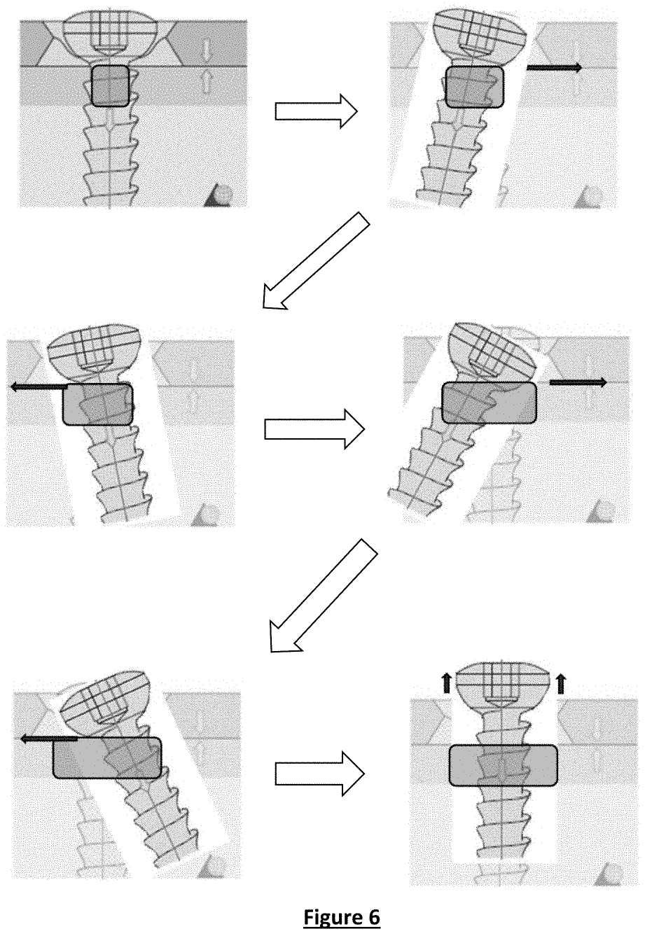

FIG. 6 depicts an exemplary series of plate and screw movements relative to a bone plate that can result in screw toggle and eventual screw “pullout” from the plate due to motion at the plate/bone interface. Desirably, the improved fixation between the bone plate and the underlying bone provided by the present invention will minimize and/or eliminate such movements.

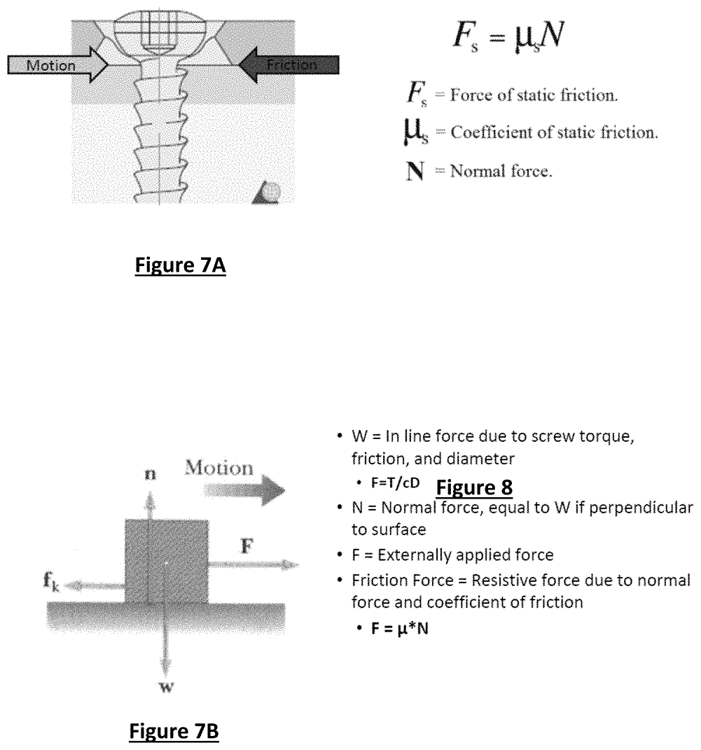

As best shown in FIGS. 7A, in conventional bone plating systems, the stability of an implant construct can depend upon (1) the normal force between the plate and the bone (which is primarily due to the screw torque or F=T/cD, and (2) the forces which resist lateral or sliding movement between the plate and bone, primarily the resistive force of friction (i.e., coefficient of friction or μ)—with such resistance being represented by the equation Fₛ=μₛN (where Fₛ is the force of static friction, μₛ is the coefficient of static friction and N is the Normal force. Referring now to FIG. 7B, these various forces will act on the bone plate in a variety of directions.

In various studies using conventional stainless steel or titanium bone plates, an average coefficient of friction between the plate and human or animal long bones has been experimentally determined to be μ=0.37, with an average torque applied to bone screws by a trauma surgeon of T=5.6 in-lb or 0.632 N-m. In many instances, this amount of torque has a limitation due to overall bone strength, and thus the Normal force from FIGS. 7A and 7B may be limited in how much force can be applied before anatomical failure. By increasing the effective coefficient of friction as described herein, however, it can be possible to dramatically improve the construct's resistant to motion relative to the bone without requiring dramatically increasing screw torque, which has been heretofore required.

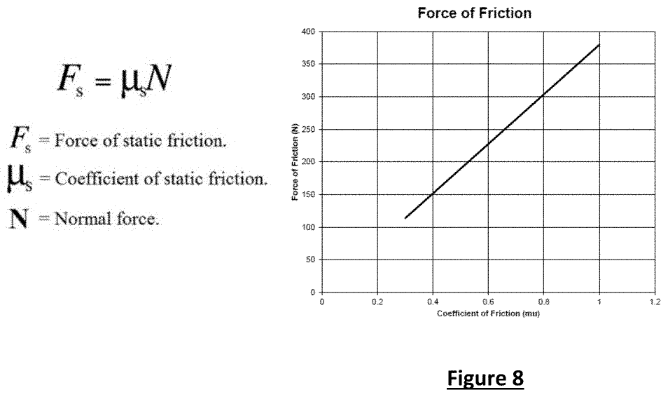

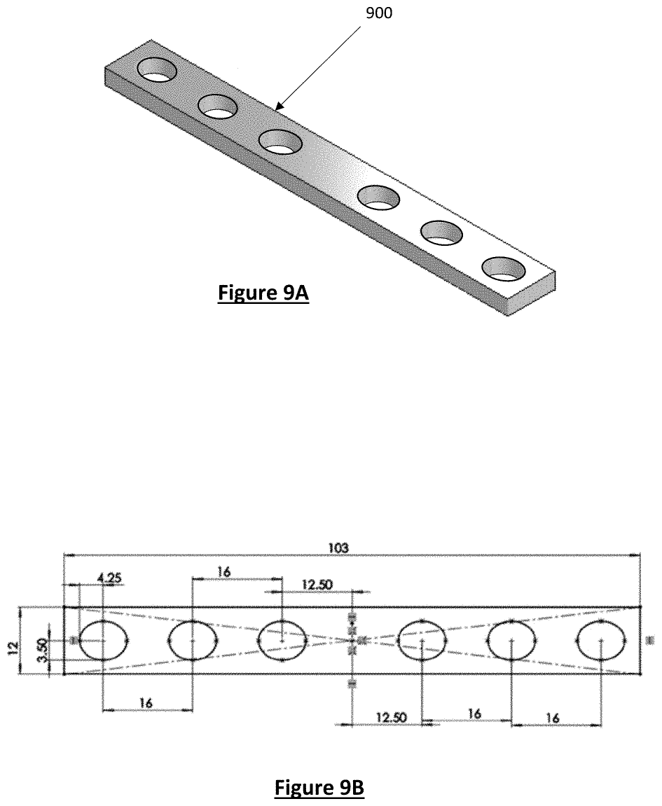

As depicted in FIG. 8, a linear relationship can exist between the Force of static friction and both the Coefficient of static friction and the Normal force. By creating a textured and/or roughened bone facing surface on the static plate 900 depicted in FIGS. 9A and 9B, therefore, the effective coefficient of friction between a textured plate and the bone surface can be dramatically increased, thereby increasing the resistance to unwanted plate/bone movement (and other effects) as described herein.

In various embodiments, a bone plate or components thereof can be formed using a variety of techniques, including by forging, casting, milling and/or extruding into sheet, tube and/or pipe form, as well as by weaving and/or braiding of component thread or “rope” into an implant by a variety of techniques. Plate shapes or surface features therein may be manufactured using subtractive manufacturing techniques (i.e., machining, milling and/or surface roughening), as well as by using additive manufacturing techniques (i.e., surface coating, brazing, welding, bonding, deposition on various material surfaces and/or even by 3D laser printing of structures). If desired, a bone plate or portions thereof may even be formed using curing or other light/energy activation techniques, such as where a slurry of liquid polymer, ceramic and/or metal particles may be UV cured to create a 3-dimensional structure and/or layer on the bone plate. In various embodiments, materials may be utilized in block form, in sheets, columns and bars, in cable or braided form, in mesh form, or in a textured surface coating, in powder form, in granular form and/or as a surface filler and/or coating. In some cases, a surface layer may be formed, placed and/or deposited on an external surface of an existing bone implant.

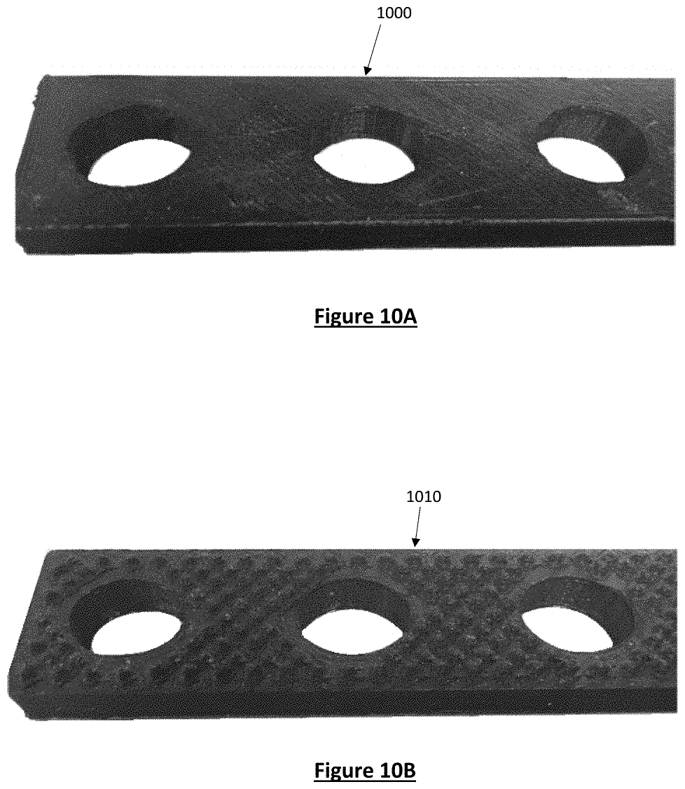

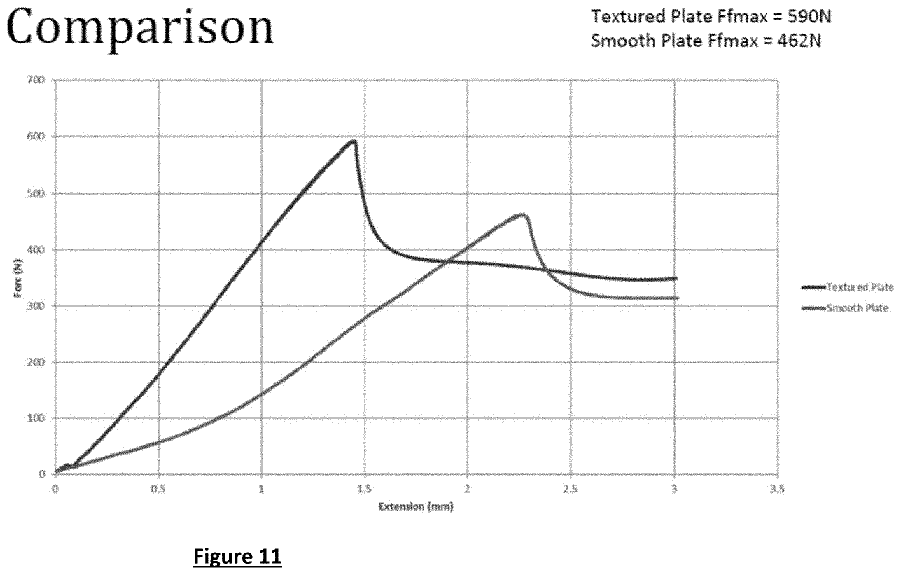

FIG. 10A depicts a “smooth” plate 1000 that has been 3D printed from a polymer precursor, while FIG. 10B depicts a textured plate 1010 having similar dimensional features which additionally incorporates a roughened bone facing surface. The employment of 3D printing of such plates can greatly enhance the utility and durability of such devices, as CAD modeling may be utilized in conjunction with 2D or 3D scans of the patient's anatomy to desirably create a bone plate having characteristics particularly useful to address the patient's unique anatomical conditions and/or surgical objectives—especially if such plate manufacture can be accomplished immediately prior to and/or during the surgical procedure. Aside from accommodating the unique anatomy of an individual patient, a wide variety of plate features and/or characteristics such as plate size, shape and/or placement of fixation holes can be particularized in an individual plate design. Similarly, the location and/or type of texturing may be modified for a given patient, such as where only a portion of the plate surface is expected to contact a bony surface of the patient. In such a case, that region (or multiple regions) of the plate may include a variety of surface features, including those described herein, to desirably increased the localized securement with the bone, while other surfaces may remain smooth or non-roughened for a variety of reasons, such as to reduce a potential for irritation and/or unwanted damage to surrounding tissues.

An experimental comparison of smooth and textured bone plates similar to those depicted in FIGS. 10A and 10B was performed. In this experiment, biomechanical humerus sawbones models were utilized, with bone fractures created using a hand saw. The test bone plates were attached to the bone models using stainless steel screws secured with a torque of 20 in-lbs, and an Instron Testing Machine was tasked to pull a constantly increasing load on the ends of the bone models, with each experiment stopped at a point where gross motion of the fracture site was observed visually.

FIG. 11 graphically depicts the motion resistance of the textured plate and smooth plates on a similar bone surface analog. More specifically, the textured plate resisted significantly more force than the smooth plate (i.e., 590N versus 462N). Moreover, the steeper curve of the textured plate as compared to the smooth plate demonstrated that the textured plate also allowed significantly less displacement or extension across the bone fracture for a given applied load (as compared to the smooth plate)—thus the textured plate could withstand significantly greater loading than the smooth plate for a variety of conditions. As significant movement across the fracture site could dramatically interfere with bone healing as well as could cause significant patient pain, this greater resistance to bone movement was deemed a significant improvement over the standard smooth plates currently available.

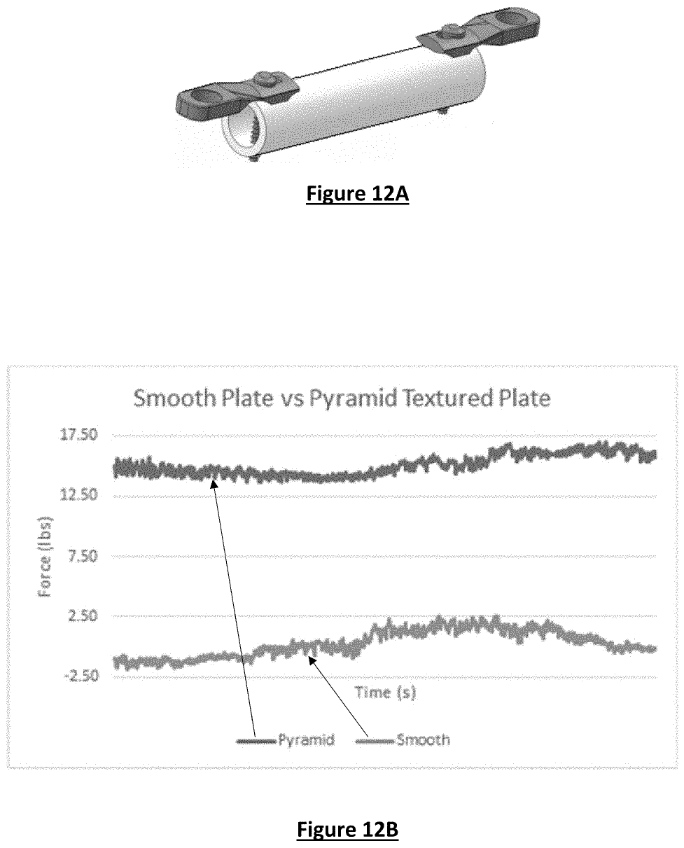

FIG. 12A depicts another embodiment of textured plates, for dynamic tensile testing purposes, one hole plates were used and secured to a test fixture. FIG. 12B graphically depicts the results of this dynamic testing model, which demonstrated that a textured plate requires significantly more force to achieve the same amount of displacement as compared to a non-textured plate. In this example, 2 mm of displacement was set as the required displacement, and the force required to achieve this displacement over many cycles was recorded. This example was done using a “pyramid” type texture in the test construct of FIG. 12A.

In various embodiments, features of the disclosed invention could be incorporated into virtually any type of plate currently used to achieve bony stability, including traditional compression plates, limited contact compression plates, locking plates, spine plates such as anterior vertebral plates, reconstruction plates, etc. It is believed that almost any bone contacting plate or implant could potentially benefit from the higher force of resistive friction provided by the present invention, and such improvements to existing device designs are contemplated herein.

In various embodiment, cervical spine plates could be utilized with texture throughout the entirety of the bone-facing side of the plate or only on the portions of the plate that attach to the vertebral body, such as areas proximate to bony fixation devices such as bone screws or similar fasteners. In addition, a lordotic curve may optionally be built into the sagittal dimensions of the plate, or the plate could have a neutral lordosis for in-situ bending by the surgeon. In addition, various fixation devices such as bone screws can be self-tapping, self-drilling, or require drill and/or tap and be fixed angle or variable angle bone screws. The screws can be able to be locked into the plate or not locked and angled towards the midline in various angles of the axial plane, from 1 to 7 degrees, depending upon the axial curvature. In addition, the plate can either be statically fixed to the vertebrae or allow some dynamic change in the length of the plate to permit some graft or intervertebral fusion device settling. Whether static or dynamic, texture could be applied in the same fashion, along the bone facing surface of the plate.

It should also be understood that the specific materials comprising the disclosed bone plates may not be critical in some embodiments, as these improvements may be useful in devices comprising materials such as stainless steel, titanium, cobalt chromium, as well as newer materials such as amorphous metals and bulk metallic glass. Virtually any implant material may have the potential to be improved as described herein, and some materials may further incorporate some or all of the benefits described herein due to ease of manufacturing in order to achieve optimal friction patterning.

Desirably, the enhancement of the coefficient of friction between the plate and bone by changing the surface of the plate that is contacting the bony surface will greatly enhance the resulting force of resistive friction, both statically and dynamically. Many possible textures or surface features, including external projections such as “pyramid type” points, ridges, chevrons, treads, etc., may significantly improve such friction, while other patterns such as inward facing or “negative” patterns (i.e., inward or “relief” type patterns machined into a bone-facing surface of the implant) may similarly be useful in various embodiments, and these patterns are contemplated herein as well.

While the plate to bone fixation herein is described in conjunction with screw fixation, it should be understood that various other fixation modalities may be utilized with the various embodiments described herein, including the use of other fixation devices (i.e., pins, staples, etc.) as well as adhesives and/or non-penetrating securement mechanism such as clamps, etc. In various embodiments, the disclosed screws may function in the same way as existing plate and screw fixation constructs by achieving unicortical or bicortical compression through torque, translating to a normal force between the plate and bone to “squeeze” these two materials together. Compression of the plate to the bone may also be achieved by clamps in certain fixation scenarios, which could then be secured by traditional non-locking or locking screws.

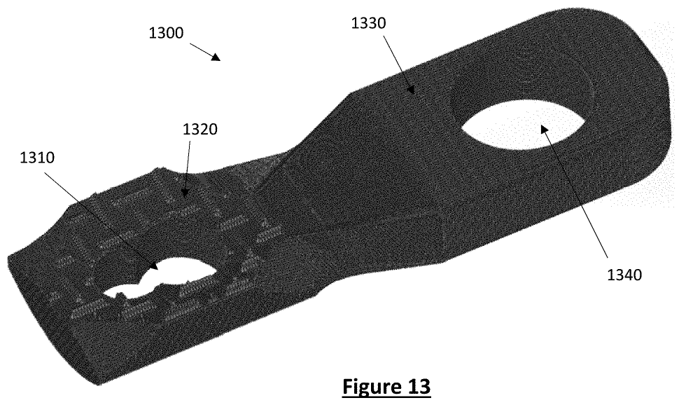

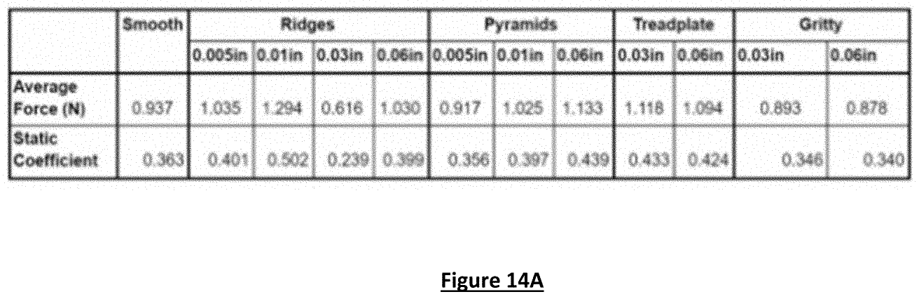

FIG. 13 depicts one exemplary embodiment of a bone plate analog for dynamic tensile testing purposes 1300 having a screw hole 1310 formed similar to holes of a convention limited contact dynamic compression plate (LC-DCP), wherein a surface of the plate proximate to the screw hole 1310 incorporates a textured surface 1320 in accordance with various teachings of the present invention. In use, this textured surface would desirably be drawn into intimate contact with the bone surface by a screw (not shown) extending through the screw hole 1310 into the bone. Bone plates of various lengths, screw hole number, sizes, and patterns could be derived from this embodiment.

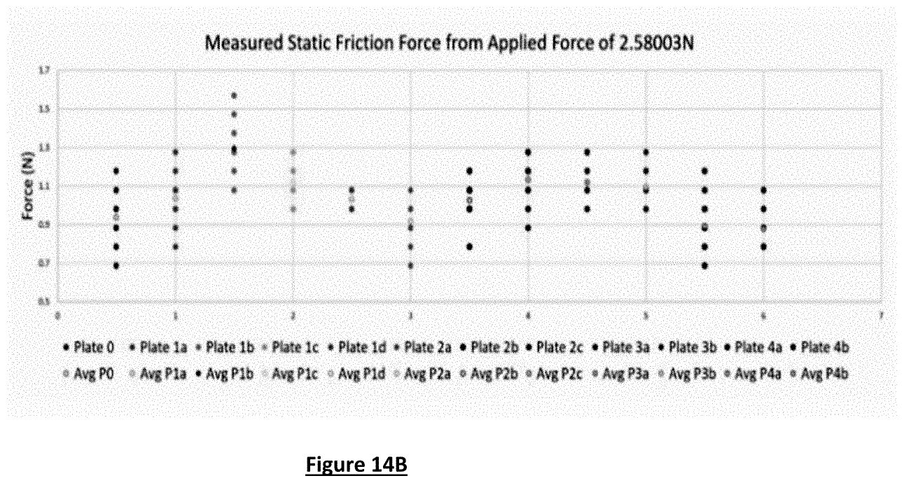

FIGS. 14A and 14B depict several different surface texture designs which were developed and constructed and were then tested against equivalent sized smooth plates to determine values for a coefficient of friction. From this testing, it appeared that pyramid shapes of 0.01 inch in size performed optimally for the proposed plate design, although further optimization of various designs might show additional improvements in various areas. The patterns analyzed included ridged shapes, pyramid shapes, treat shapes and grit shapes.

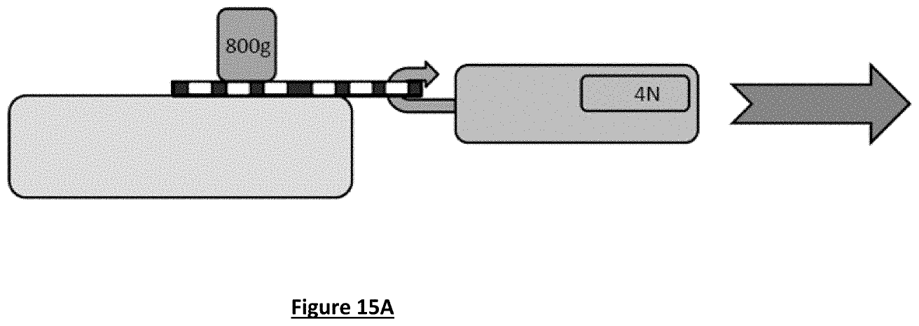

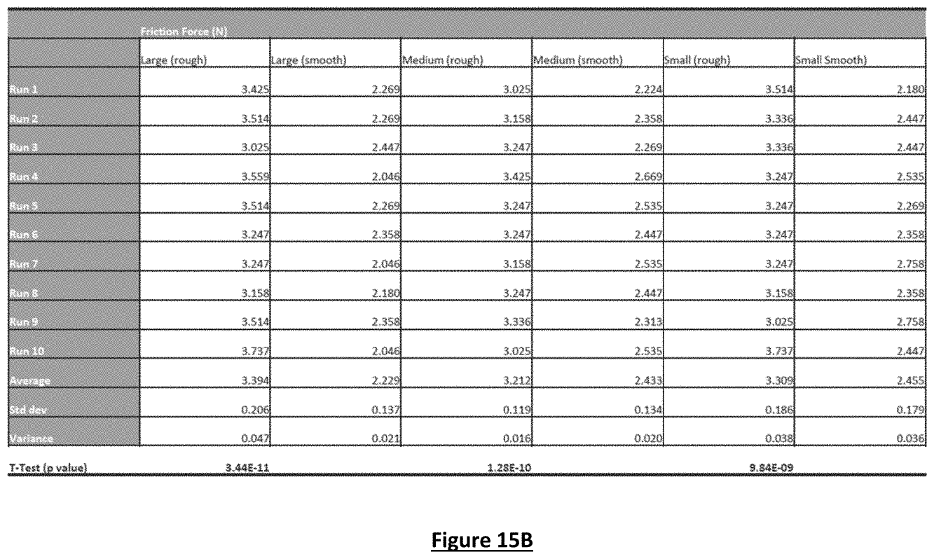

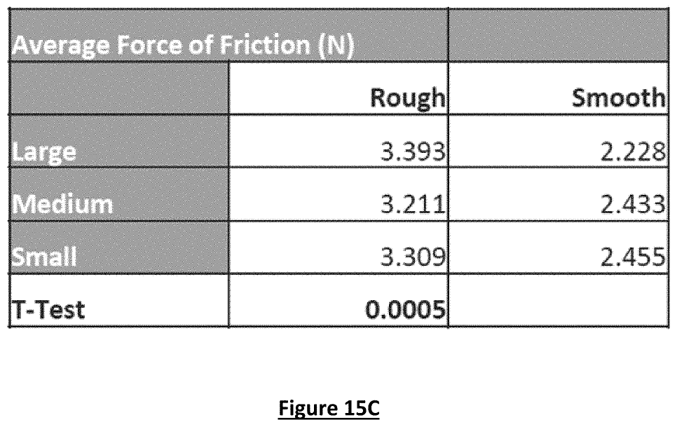

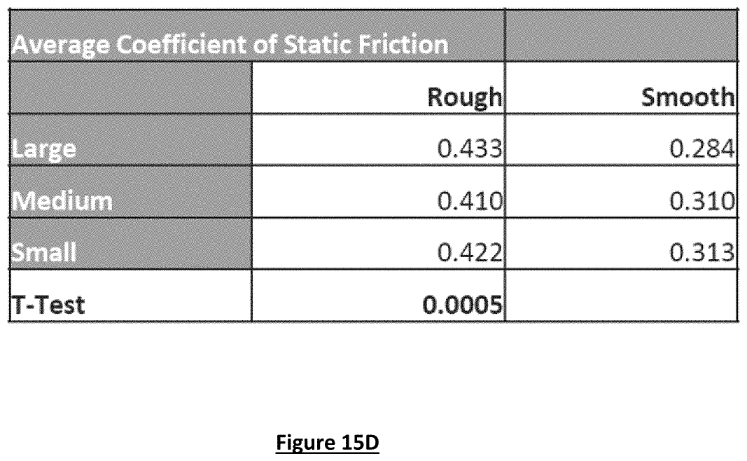

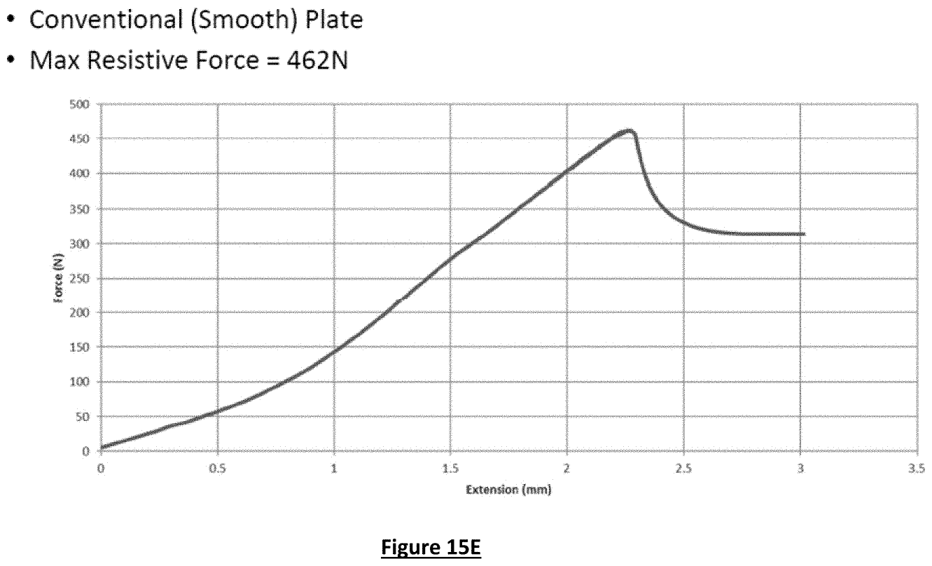

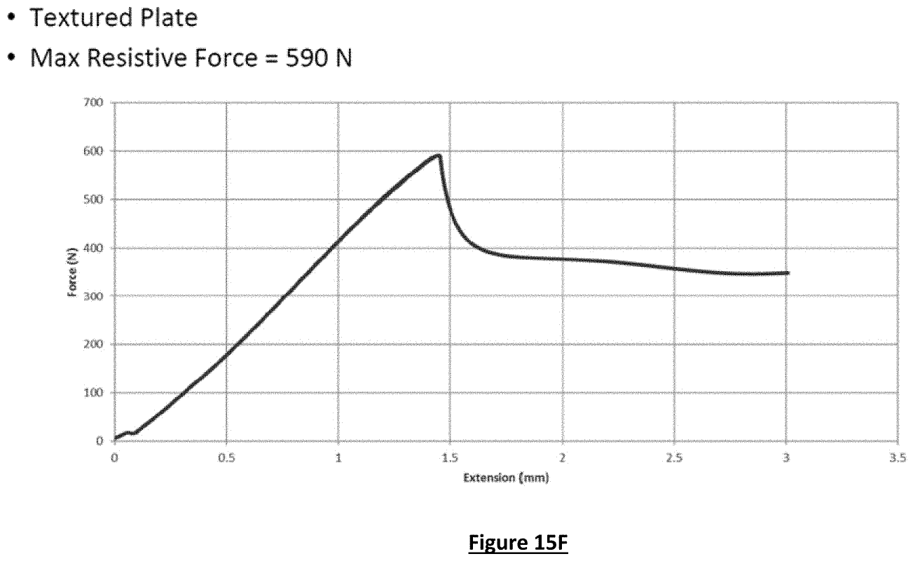

FIG. 15A depicts another exemplary experiment comparing plates with the addition of bone facing textured surfaces against smoother surfaced plates, wherein 6 plates were designed and manufactured (i.e., 3D printed), with 3 of the plate sets being textured (i.e., 100, 200 and 300% designed size) and three of the plate sets being smooth (i.e., 100, 200 and 300% designed size). The experimental design was to apply an eight hundred (800) gram weight on a top surface of the 3D printed plate and measuring a Friction force using a tensiometer while applying a 4N lateral load, with 10 runs per plate. An analysis on the force of friction as well as the coefficient of friction was conducted. FIG. 15B graphically depicts various results from the experiment of FIG. 15A.

Statistical analysis of this experimental data was performed with a 2-tailed T-test, which demonstrated that roughening and/or texturing the bone contact surface of the plate had a significant impact on increases of the coefficient of friction, and also on increased force of friction (See FIGS. 15C and 15D). A graphic depiction of these results in provided in FIGS. 15E and 15F.

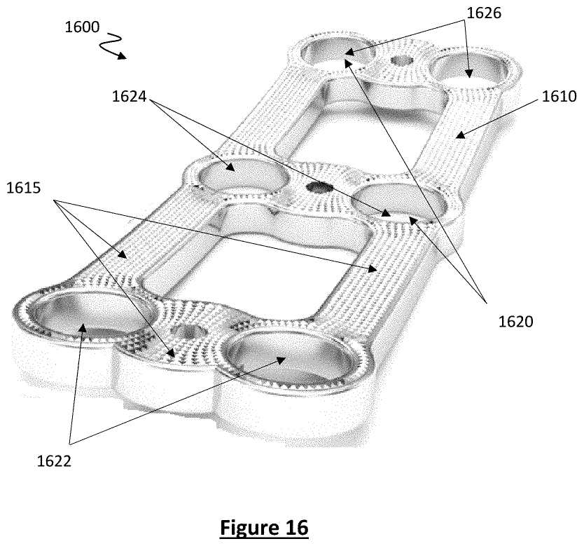

FIG. 16 depicts another exemplary embodiment of a surgical plate 1600 for use with various surgical procedures. In this embodiment the plate 1600 can include a bone-facing surface 1610 which incorporates a plurality of protrusions 1615 or other surface features which desirably increase frictional and/or mechanical interlocking forces between the plate 1600 and the underlying bone (not shown). In this embodiment, the protrusions 1615 can comprise a series of repeating geometric patterns, such as cones or four or three sides pyramids, as previously described. The plate also includes a plurality of openings 1620 which accommodate bone screws (not shown) or other fixation devices, which desirably anchor and/or secure the plate against one or more underlying bony surfaces. In the disclosed plate 1600, a first set 1622 of openings 1620 can attached to a superior cervical vertebrae, a second set 1624 of openings 1620 can attached to an intermediate cervical vertebrae, and/or a third set 1626 of openings 1620 can optionally attached to an inferior cervical vertebrae, if desired.

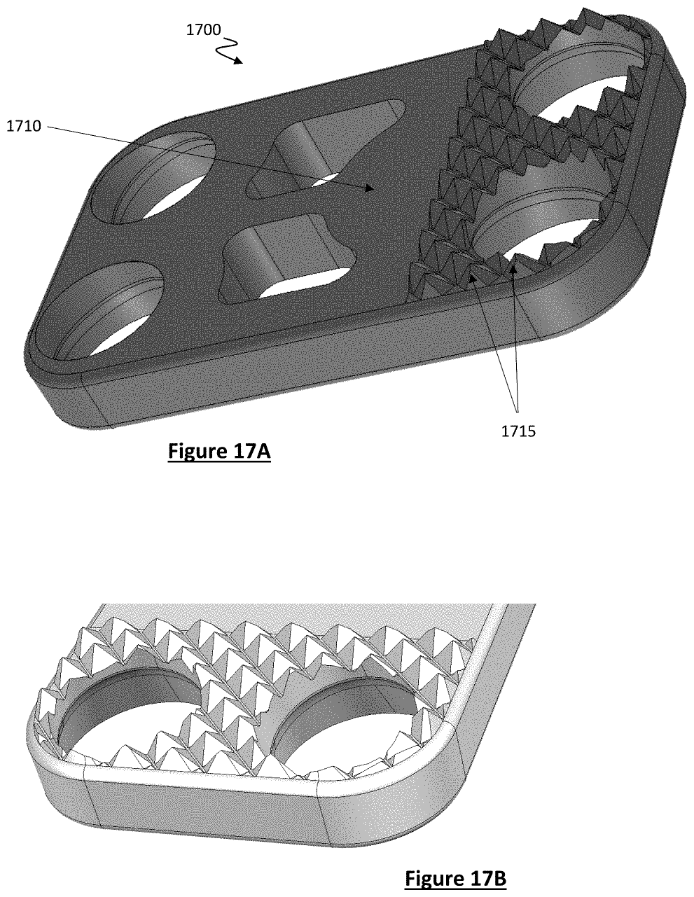

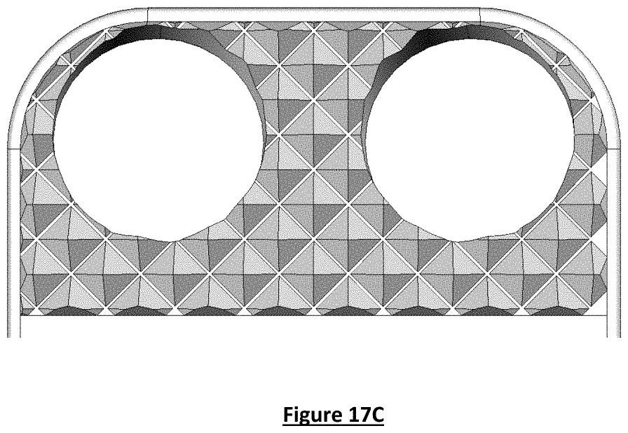

FIG. 17A depicts another alternative embodiment of a surgical plate 1700 for use with various surgical procedures. In this embodiment the plate 1700 includes a bone-facing surface 1710 which incorporates a plurality of protrusions 1715 or other surface features which desirably increase frictional and/or mechanical interlocking forces between the plate 1700 and the underlying bone. In this embodiment, the protrusions 1715 comprise a series of repeating 4-sided pyramid shapes, which are relatively closely packed (see FIGS. 17B and 17C). As best seen in FIG. 17C, the protrusions can be symmetrically distributed over the plate surface, although in other embodiment such distribution could be asymmetrical and/or substantially random. FIG. 17C also depicts the limited spacing between adjacent protrusions, which in this embodiment are significantly less (i.e., less than 20% or less than 10%) of the base width of the protrusion, with adjacent upward points or vertices of the protrusions spaced apart from each other by slightly more than the base width of each protrusion.

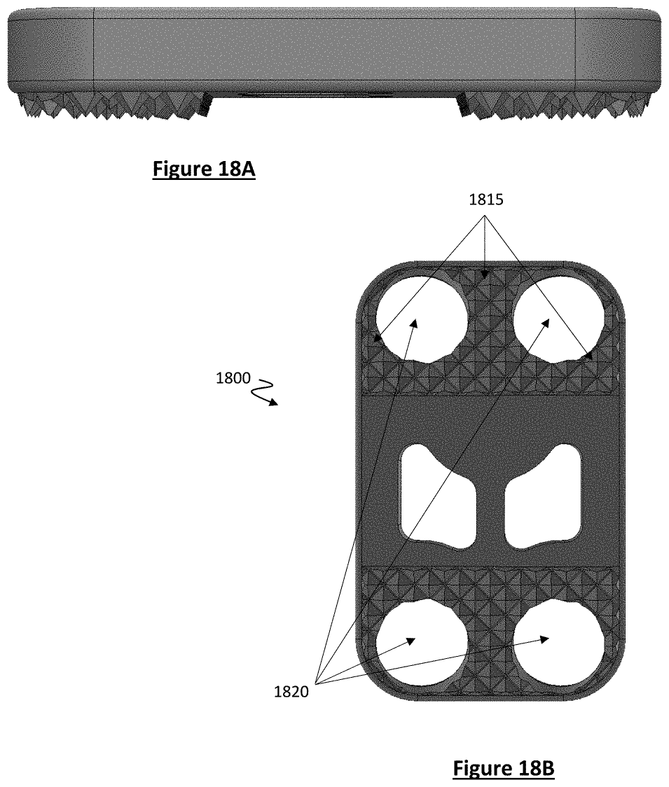

FIGS. 18A and 18B depict views of another alternative embodiment of a surgical plate 1800 for use with various surgical procedures, wherein the protrusions 1815 are distributed around each of the fixation openings 1820 of the plate 1800.

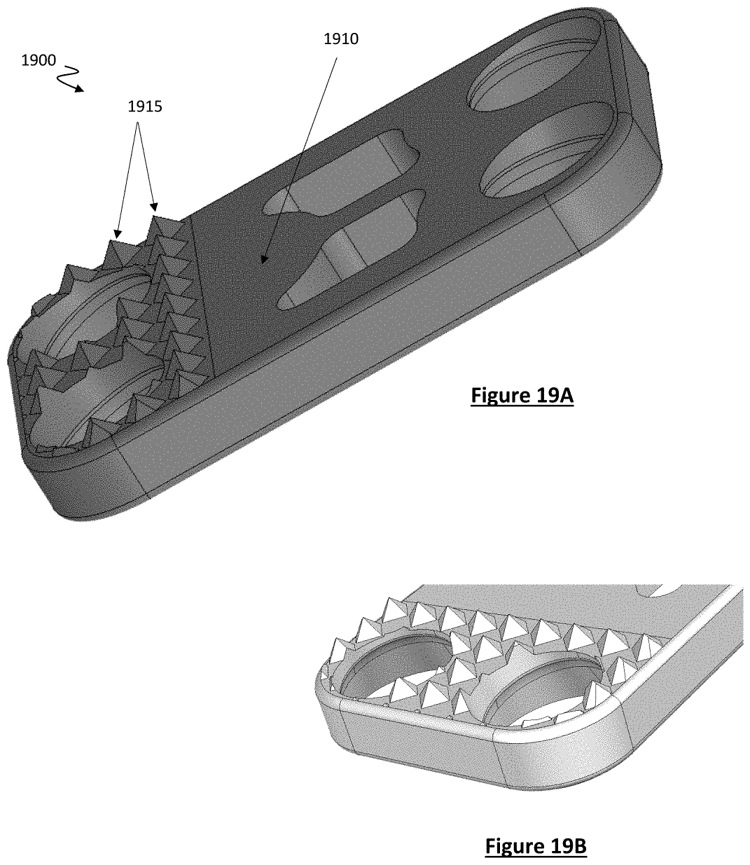

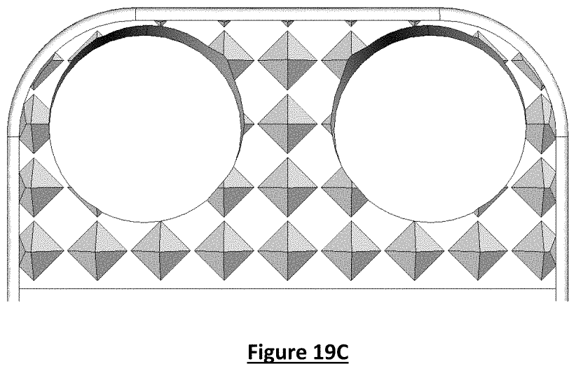

FIGS. 19A through 19C depict views of another alternative embodiment of a surgical plate 1900 for use with various surgical procedures. In this embodiment the plate 1900 includes a bone-facing surface 1910 which incorporates a plurality of protrusions 1915 or other surface features which desirably increase frictional and/or mechanical interlocking forces between the plate 1900 and the underlying bone. In this embodiment, the protrusions 1915 comprise a series of repeating 4-sided pyramid shapes, which are spaced apart (see FIGS. 19B and 19C). The protrusions may be symmetrically distributed over the plate surface, although in other embodiments such distribution could be asymmetrical and/or substantially random. FIG. 19C also depicts the expanded spacing between adjacent protrusions, which in this embodiment are significantly equal to or greater than the base width of the protrusion, with adjacent upward points or vertices of the protrusions paced apart from each other by at least twice the width of each protrusion in some directions (and spaced apart from each other by slightly more than the base width of each protrusion in at least one other direction).

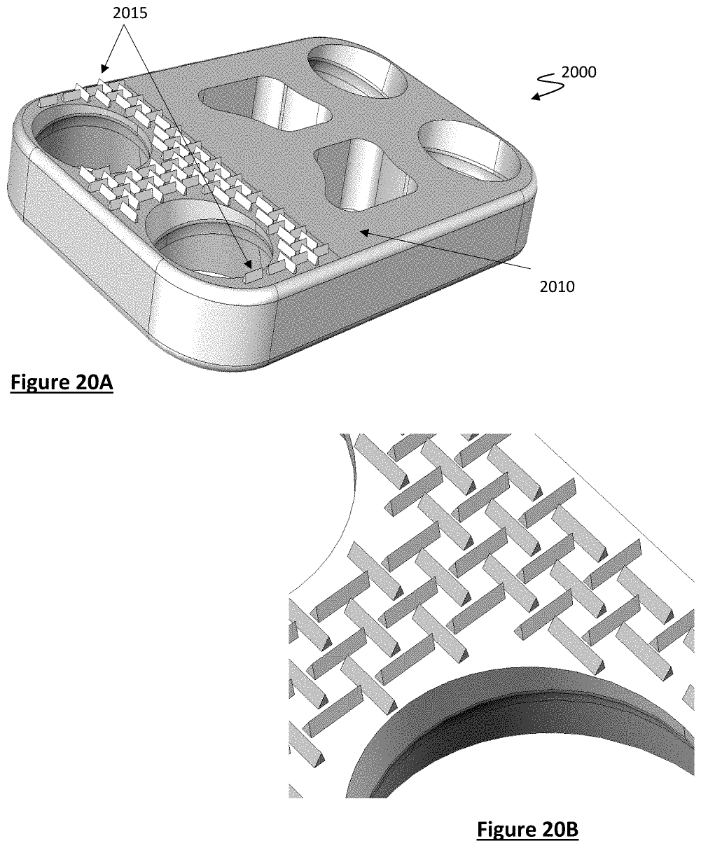

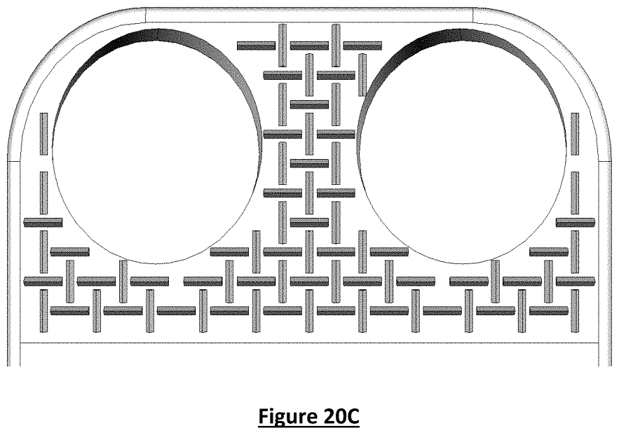

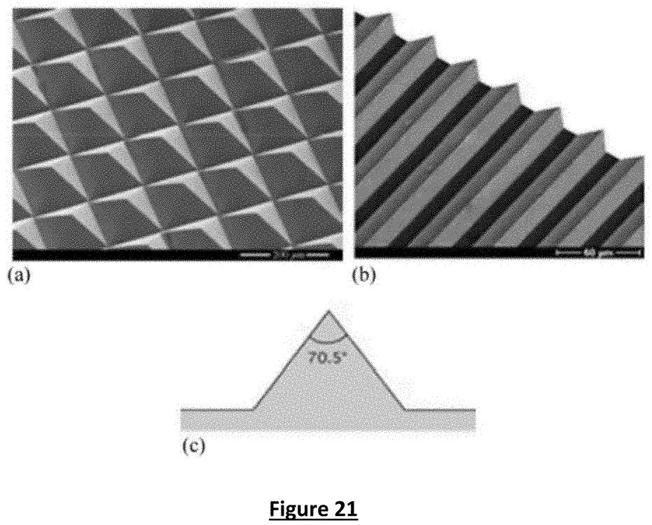

FIGS. 20A through 20C depict view of another alternative embodiment of a surgical plate 2000 for use with various surgical procedures. In this embodiment the plate 2000 includes a bone-facing surface 2010 which incorporates a plurality of protrusions 2015 or other surface features which desirably increase frictional and/or mechanical interlocking forces between the plate 2000 and the underlying bone. In this embodiment, the protrusions 2015 comprise a series of elongated tread-shapes, which are spaced apart and distributed in alternating repeating rows (see FIG. 20C). The protrusions may be symmetrically distributed over the plate surface, although in other embodiments such distribution could be asymmetrical and/or substantially random. FIG. 20C also depicts the packing arrangement of adjacent protrusions, which in this embodiment have one end of a protrusion directly adjacent to a midpoint of an alternating adjacent protrusion.

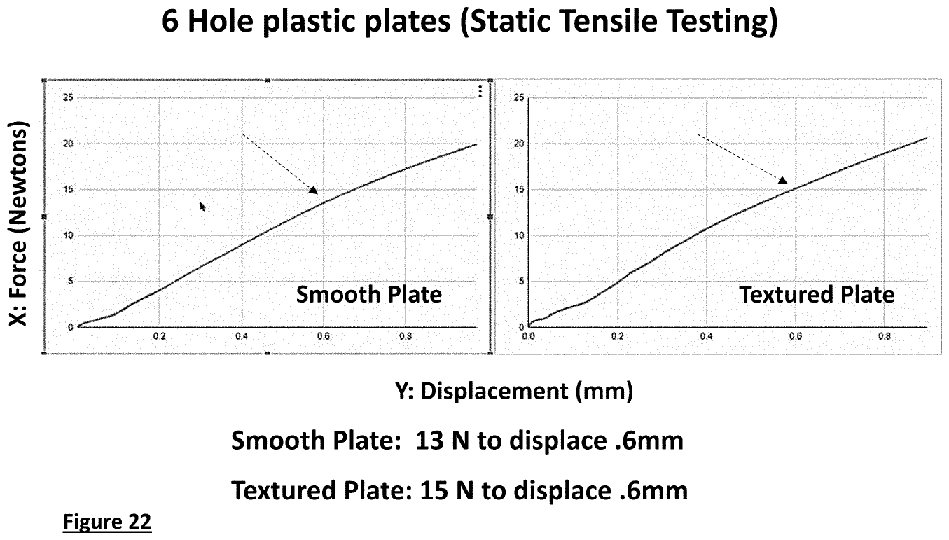

FIG. 21 depicts exemplary embodiments of (a) pyramidal-shaped protrusions and (b) elongated wave or tread-shaped protrusions. In both of these embodiments, an upper tip or vertex of the protrusion desirably can include a relatively sharp corner or plane, with the sides forming the protrusion positioned at an angle of approximately 70.5° (degrees), as shown in (c). In various embodiments, the tip of the protrusion will desirably form a point or knife edge that is sufficiently acute to penetrate bony tissue and/or substantially increase the localized resistance to sliding—i.e., the localized coefficient of friction between the plate and bone and/or mechanical interlocking with various bone structures. In some embodiments the sides forming the tip of the protrusion may form an apex with an internal acute angle ranging from 30° (degrees) to 90° (degrees) and/or up to 145° (degrees). In various embodiments, the apex angle may comprise an angle less than 75 degrees, while in other embodiment the apex angle may be greater than 75 degrees, while in still other embodiments the apex angle may range from 60 degrees to 120 degrees, depending upon application and underlying bone type and/or quality. Protrusions may include 2-sided tread forms, or 3 or 4 or more sided projections such as pyramidal shapes, if desired.

FIG. 22 depicts results from an exemplary comparison between a smooth plastic plate and a textured plastic plate, where the resistance of the textured plastic plate to lateral movement is greatly increased by texturing or roughening the surface of the plate, wherein the textured plate requires over 15% more force to laterally displace as compared to an identical smooth plate of similar construction.

Desirably, the disclosed surface enhancement of orthopedic surgical plates and/or similar devices will increase the securement due to the force of resistive friction at the bone/plate interface in order to create a more stable construct and greatly reduce wear to and/or fracture of the system components. In addition to allowing the plate to accommodate higher loading, the present invention can significantly reduce toggle and/or fracture of the bone screws—greatly enhancing the utility of the disclosed devices and well as existing system forces that may be modified in accordance with various teachings of the present invention.

In various embodiments, a proposed design may include the creation of a “textured” surface proximate to a securement device (i.e., bone screw) in order to enhance the coefficient of friction between the plate and the bone near that location in order to limit motion and increase stability of various fixation constructs.

In various embodiments, the implants and/or portions may comprise a variety of surgically acceptable materials, including radiopaque and/or radiolucent materials, other materials or combinations of such materials. Radiolucent materials can include, but are not limited to, polymers, carbon composites, fiber-reinforced polymers, plastics, combinations thereof and the like. Radiopaque materials are traditionally used to construct devices for use in the medical device industry. Radiopaque materials can include, but are not limited to, metal, aluminum, stainless steel, titanium, titanium alloys, cobalt chrome alloys, combinations thereof and the like.

In accordance with various aspects of the present subject matter, bone-contacting surfaces of the various implant components described herein may optionally include coatings that are highly osteo-inductive and/or osteoconductive to desirably facilitate and/or promote fixation to adjacent living bone surfaces, if desired.

If desired, a surgical “kit” could include bone plate components of differing shapes and/or sizes constructed from the same or a variety of different materials, including plates having a variety of different bone anchors and/or interface surfaces, including bone ingrowth surfaces. If desired, such implants and/or the components thereof could be provided in a kit form for selection and/or assembly in situ during a surgical procedure. If desired, various components may be removable and/or replaceable.

In accordance with another aspect of the present subject matter, various methods for manufacturing implants and/or components thereof, as set for within any of the details described with the present application, may be provided.

While embodiments and applications of the present subject matter have been shown and described, it would be apparent that other embodiments, applications and aspects are possible and are thus contemplated and are within the scope of this application.

In various embodiments, a surgical tool kit could include a plurality of bone plate implants and one or more modular components for the system. The various components of these systems could optionally be provided in kit form, with a medical practitioner having the option to select an appropriately sized and/or shaped implant and/or modular components to address a desired surgical situation.

Note that, in various alternative embodiments, variations in the position and/or relationships between the various figures and/or modular components are contemplated, such that different relative positions of the various modules and/or component parts, depending upon specific module design and/or interchangeability, may be possible. In other words, different relative adjustment positions of the various components may be accomplished via adjustment in separation and/or surface angulation of one of more of the components to achieve a variety of resulting implant configurations, shapes and/or sizes, thereby accommodating virtually any expected anatomical variation.

Of course, method(s) for manufacturing the various modular components and/or surgical devices and related components and implanting a bone plate into a patent are contemplated and are part of the scope of the present application.

While embodiments and applications of the present subject matter have been shown and described, it would be apparent to those skilled in the art that many more modifications are possible without departing from the inventive concepts herein. The subject matter, therefore, is not to be restricted except in the spirit of the appended claims.

All references, including publications, patent applications, and patents, cited herein are hereby incorporated by reference to the same extent as if each reference were individually and specifically indicated to be incorporated by reference and were set forth in its entirety herein.

The various headings and titles used herein are for the convenience of the reader and should not be construed to limit or constrain any of the features or disclosures thereunder to a specific embodiment or embodiments. It should be understood that various exemplary embodiments could incorporate numerous combinations of the various advantages and/or features described, all manner of combinations of which are contemplated and expressly incorporated hereunder.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., i.e., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventor for carrying out the invention. Variations of those preferred embodiments may become apparent to those of ordinary skill in the art upon reading the foregoing description. The inventor expects skilled artisans to employ such variations as appropriate, and the inventor intends for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A surgical plate capable of fixation between adjacent vertebral bodies using a plurality of bone screws, comprising:
an elongated plate body having a first bone facing side and an opposing second side, a plurality of screw openings formed through the elongated plate body from the first bone facing side to the opposing second side, a portion of the first bone facing side proximate to at least one of the plurality of openings including a first textured surface.

2. The surgical plate of claim 1, wherein the first textured surface comprises a plurality of projections extending outward from the elongated plate body.

3. The surgical plate of claim 2, wherein the plurality of projections increases the static coefficient of friction of the first textured surface by at least 15% as compared to a smooth surface of a similar size.

4. The surgical plate of claim 2, wherein each of the plurality of projections includes a distal tip portion, and at least a portion of the distal tip portions of the plurality of projections penetrates an outer surface of at least one of the adjacent vertebral bodies.

5. The surgical plate of claim 2, wherein the plurality of projections increases the static coefficient of friction of the first textured surface by at least 50% as compared to a smooth surface of a similar size.

6. The surgical plate of claim 2, wherein each of the plurality of projections comprise a pyramid shape having an apex angle of approximately 70.5 degrees.

7. The surgical plate of claim 2, wherein each of the plurality of projections comprise a ridged shape having an apex angle of less than 75 degrees.

8. The surgical plate of claim 2, wherein each of the plurality of projections comprise a treadplate.

9. The surgical plate of claim 2, wherein the plurality of projections are distributed uniformly across the first textured surface.

10. The surgical plate of claim 2, wherein the plurality of projections are distributed symmetrically across the first textured surface.

11. The surgical plate of claim 2, wherein the plurality of projections are non-uniformly distributed across the first textured surface.

12. The surgical plate of claim 2, wherein the plurality of projections are non-uniformly distributed across the first textured surface.

13. The surgical plate of claim 1, wherein the portion of the first bone facing side includes a second textured surface proximate to a second of the plurality of openings, the second texture surface having a different bone facing surface configuration than the first textured surface.

14. The surgical plate of claim 2, wherein the plurality of projections are formed on the elongated plate using 3-D printing techniques.

15. The surgical plate of claim 2, wherein the plurality of projections are formed on the elongated plate by subtractive machining techniques.

16. A method of improving a coefficient of friction between a bone facing surface of a surgical plate and an anterior surface of a cervical bone against which the plate is implanted, the method comprising the steps of:
altering a surface finish on the bone facing surface by forming a plurality of projections extending outward of the bone facing surface, the plurality of projection increasing the effective coefficient of static friction of the bone facing surface with the anterior surface by at least 10%;
securing the surgical plate against the anterior surface using a plurality of fixation devices.

17. The method of claim 16, wherein the plurality of fixation devices comprises a plurality of bone screws, each of the plurality of bone screws penetrating the anterior surface of the bone.

18. The method of claim 17, wherein each of the plurality of bone screws extends through one of a plurality of openings extending through the surgical plate, the plurality of projections formed on the bone facing surface at a location proximate to at least one of the plurality of openings extending through the surgical plate.

19. The method of claim 18, wherein the step of altering a surface finish on the bone facing surface comprises altering the surface finish on the bone facing surface by adding material to the bone facing surface without significantly altering the overall dimensions or rigidity of the surgical plate.

20. The method of claim 18, wherein the step of altering a surface finish on the bone facing surface comprises altering the surface finish on the bone facing surface by removing material from the bone facing surface without significantly altering the overall dimensions or rigidity of the surgical plate.
